(19) United States
(12) Patent Application Publication
(10) Pub. No.: US 2016/0038117 A1
(43) Pub. Date: Feb. 11, 2016

(54) ULTRASONIC BLOOD PRESSURE MEASURING DEVICE AND BLOOD PRESSURE MEASURING METHOD

(71) Applicant: SEIKO EPSON CORPORATION, Tokyo (JP)

(72) Inventor: Natsumi TAMADA, Shiojiri-shi (JP)

(21) Appl. No.: 14/820,877

(22) Filed: Aug. 7, 2015

(30) Foreign Application Priority Data
Aug. 11, 2014 (JP) 2014-163412

(51) Int. Cl. A61B 8/04; A61B 8/08

(52) U.S. Cl. CPC A61B 8/04; A61B 8/5223

(57) ABSTRACT

An ultrasonic blood pressure measuring device measures the blood pressure of a blood vessel by performing transmission of an ultrasonic wave and reception of a reflected wave with respect to the blood vessel using an ultrasonic probe. A blood vessel position determination unit determines the position of the blood vessel with respect to the ultrasonic probe based on the reception signal of the ultrasonic probe. A blood pressure measurement execution unit executes blood pressure measurement based on the relative position of the blood vessel determined by the blood vessel position determination unit.

### BACKGROUND

1. Technical Field

The present invention relates to an ultrasonic blood pressure measuring device and the like for performing blood pressure measurement using an ultrasonic wave.

2. Related Art

In recent years, a non-invasive blood pressure measuring method using an ultrasonic wave has been proposed as a method of measuring blood pressure in a non-pressure manner without using a cuff.

For example, a method of regarding a blood vessel diameter change and a blood pressure change in a local part as a non-linear function and calculating the blood pressure from a stiffness parameter β indicating the hardness of a blood vessel and the diameter of the blood vessel has been proposed in JP-A-2004-41382. According to this method, it is possible to perform continuous blood pressure monitoring for each heart beat without forcing a burden on a patient due to the tightening of a cuff unlike in the related art.

In order to realize the non-invasive and continuous blood pressure measurement, it is desirable to always attach an ultrasonic sensor to a part to be measured. However, this is considerably affected by the posture change of the body. For example, assuming that the carotid artery is a part to be measured, when the direction of the head is changed up and down or left and right by the movement of the neck, the carotid artery expands and contracts in the long axis direction. In this case, the thickness of the blood vessel wall may change, or the hardness of the blood vessel may change from that at the time of calibration. For this reason, an error may be included in the blood pressure value that is calculated from the relationship between the blood pressure and the blood vessel diameter. Since the error allowed for the measurement accuracy of the blood vessel diameter may be several micrometers to tens of micrometers, the measurement accuracy of the blood vessel diameter is important.

### SUMMARY

An advantage of some aspects of the invention is to provide a technique capable of eliminating the influence of the posture change of a person to be measured during blood pressure measurement.

A first aspect of the invention is directed to an ultrasonic blood pressure measuring device that measures blood pressure of a blood vessel by performing transmission of an ultrasonic wave and reception of a reflected wave with respect to the blood vessel using an ultrasonic probe, the device including: a blood vessel position determination unit that determines a position of the blood vessel with respect to the ultrasonic probe based on a reception signal of the reflected wave; and a blood pressure measurement execution unit that executes blood pressure measurement based on a determination result of the blood vessel position determination unit.

According to the first aspect of the invention, it is possible to manage the execution of measurement by determining the position of the blood vessel to be measured with respect to the ultrasonic probe. In other words, when there is a relative positional relationship, for example, when an error is included in the measurement result, no measurement can be performed. Therefore, it is possible to realize non-invasive blood pressure measurement with high measurement accuracy by eliminating the influence of the posture change of a person to be measured. When performing continuous measurement, it is possible to perform no measurement in a state in which the body posture of the person to be measured causes a measurement error but to repeat measurements when the person to be measured has an appropriate posture.

A second aspect of the invention is directed to the ultrasonic blood pressure measuring device according to the first aspect of the invention, wherein the blood vessel position determination unit determines the position of the blood vessel according to a center of the blood vessel.

As elements representing the blood vessel position, the front wall (blood vessel wall on a side close to the ultrasonic probe) or the rear wall of the blood vessel can also be used. However, the blood vessel wall continuously expands and contracts due to beating. In contrast, there is no such influence on the center of the blood vessel. Therefore, it is possible to maintain high measurement accuracy.

A third aspect of the invention is directed to the ultrasonic blood pressure measuring device according to the first or second aspect of the invention, wherein the blood pressure measurement execution unit executes blood pressure measurement when the position is included in a measurement allowable blood vessel position range set in advance.

From the viewpoint of measurement accuracy, if the posture of the person to be measured is completely the same as the posture when calculating the relationship between the blood pressure and the blood vessel diameter, there is no possibility that an error will occur. However, it is difficult to completely reproduce the same posture. In addition, a situation may also occur in which measurement is not performed even though continuous measurement is scheduled. However, according to the third aspect of the invention, it is possible to set an appropriate allowable range for determining the position of the blood vessel. By setting the measurement allowable blood vessel position range, it is possible to ensure practicability while allowing the error range that does not cause any problems in practice.

A fourth aspect of the invention is directed to the ultrasonic blood pressure measuring device according to the third aspect of the invention, which further includes a notification control unit that performs predetermined notification control when the position is outside of the measurement allowable blood vessel position range.

According to the fourth aspect of the invention, it is possible to notify the operator of a situation where an error is included in measurement due to a posture change. Through this notification, it is possible to prompt the person to be measured to correct their posture. When measurement is performed even though the position of the blood vessel is outside of the measurement allowable blood vessel position, there may be a misunderstanding that the device may have failed. The misunderstanding is resolved by the notification.

A fifth aspect of the invention is directed to the ultrasonic blood pressure measuring device according to the third or fourth aspect of the invention, the blood vessel may be a carotid artery, and the measurement allowable blood vessel position range may be set such that a range of the position in a direction crossing a transmission direction of the ultrasonic wave with respect to the ultrasonic probe is larger than a range of the position in the transmission direction of the ultrasonic wave with respect to the ultrasonic probe.

In the case of a change in the position of the carotid artery due to a change in the posture of the head, the change in the position of the carotid artery in a parallel movement direction (direction crossing the transmission direction of the ultrasonic wave) with respect to the ultrasonic probe, that is, in a direction along the arrangement of ultrasonic transducers is larger than that in the depth direction (transmission direction of the ultrasonic wave) with respect to the ultrasonic probe. Therefore, it is possible to set the measurement allowable blood vessel position range more practically.

A sixth aspect of the invention is directed to a blood pressure measuring method including: performing transmission of an ultrasonic wave and reception of a reflected wave with respect to a blood vessel using an ultrasonic probe and acquiring received data based on a reception signal of the reflected wave; determining a position of the blood vessel with respect to the ultrasonic probe based on the received data; and executing blood pressure measurement based on a result of the determination.

According to the sixth aspect of the invention, the same effect as in the first aspect of the invention is obtained.

A seventh aspect of the invention is directed to the blood pressure measuring method according to the sixth aspect of the invention, wherein in the determination, the position of the blood vessel may be determined according to a center of the blood vessel.

According to the seventh aspect of the invention, the same effect as in the second aspect of the invention is obtained.

### BRIEF DESCRIPTION OF THE DRAWINGS

The invention will be described with reference to the accompanying drawings, wherein like numbers reference like elements.

FIG. 1 is a diagram showing an example of the system configuration of an ultrasonic blood pressure measuring device.

FIG. 2 is a diagram showing an example of a change in the blood vessel position of the carotid artery with respect to an ultrasonic probe, and shows an example when shaking the head from side to side.

FIG. 3 is a diagram showing an example of a change in the blood vessel position of the carotid artery with respect to an ultrasonic probe, and shows an example when shaking the head up and down.

FIG. 4 is a diagram for explaining a method of determining the blood vessel position.

FIG. 5 is a diagram for explaining a method of determining a specific posture state based on the blood vessel position.

FIG. 6 is a block diagram showing an example of the functional configuration of the ultrasonic blood pressure measuring device.

FIG. 7 is a flowchart for explaining the flow of the process of the ultrasonic blood pressure measuring device.

FIG. 8 is a flowchart for explaining the flow in a modification example of the process of the ultrasonic blood pressure measuring device.

FIG. 9 is a diagram showing a display example of reliability information.

FIG. 10 is a flowchart for explaining the flow of the automatic setting process for automatically setting the measurement allowable blood vessel position range.

FIGS. 11A-11C are diagrams for explaining a modification example of the method of calculating the blood vessel position.

FIGS. 12A and 12B are diagrams for explaining a modification example of the method of calculating the blood vessel position.

### DESCRIPTION OF EXEMPLARY EMBODIMENTS

### First Embodiment

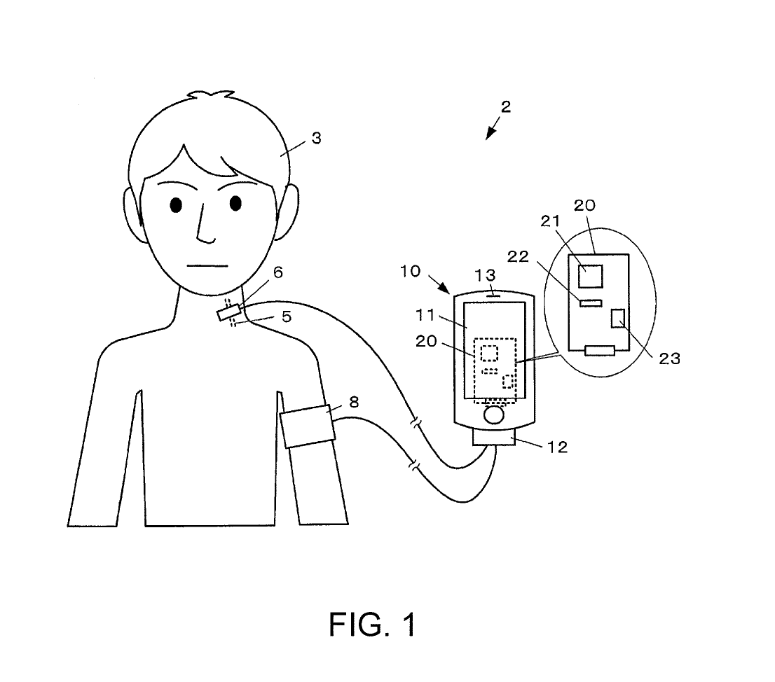

FIG. 1 is a diagram showing an example of the configuration of an ultrasonic blood pressure measuring system in the present embodiment.

An ultrasonic blood pressure measuring system 2 includes an ultrasonic probe 6 that is always attached to a part to be measured (in the present embodiment, a carotid artery 5) of a person to be measured 3, a calibration sphygmomanometer 8, and an ultrasonic blood pressure measuring device 10.

The ultrasonic probe 6 performs transmission and reception of an ultrasonic wave, generates a reception signal corresponding to the strength of the received reflected wave, and outputs the generated reception signal to the ultrasonic blood pressure measuring device 10. Such a function can be realized by the technique according to the known ultrasonic measurement.

In the present embodiment, immediately above the left carotid artery 5, at least one of a plurality of ultrasonic transducers arrayed in the ultrasonic probe 6 is attached along the long axis of the blood vessel so as to capture a short-axis image of the blood vessel. “Immediately above” referred to herein is used, for easy understanding, in the operating manual expression when operating the ultrasonic probe 6. To be precise, “immediately above” refers to the positional relationship in which the carotid artery is located on the straight line of irradiation of ultrasonic waves that are emitted from the ultrasonic transducer arrayed in the ultrasonic probe 6.

The calibration sphygmomanometer 8 measures blood pressure for calculating the relationship between blood pressure and a blood vessel diameter (hereinafter, referred to as “blood pressure-blood vessel diameter relationship”), and outputs the measurement result to the ultrasonic blood pressure measuring device 10. In the present embodiment, a cuff type electronic sphygmomanometer is used. However, other types of sphygmomanometers, such as a tonometry sphygmomanometer capable of measuring blood pressure every beat, may be used. In addition, the calibration sphygmomanometer 8 can be removed after calculating the blood pressure-blood vessel diameter relationship by performing calibration before the start of measurement.

The ultrasonic blood pressure measuring device 10 is a computer to realize a blood pressure measuring function, and realizes 1) a blood vessel position determining function for determining the relative position of the blood vessel with respect to the ultrasonic probe 6 based on the reception signal obtained from the ultrasonic probe 6, 2) a blood pressure measurement execution function for executing blood pressure measurement using the ultrasonic probe 6 based on the determination result of the blood vessel position, and 3) a data logging function for periodically executing information, such as the measured blood pressure, until the predetermined measurement end conditions are satisfied and storing the measurement result.

Specifically, the ultrasonic blood pressure measuring device 10 includes a touch panel 11, an interface circuit 12, a speaker 13, a control board 20, and an internal battery (not shown). A central processing unit (CPU) 21 executes a program stored in an IC memory 22 mounted on the control board 20, and the ultrasonic blood pressure measuring device 10 realizes the above-described blood vessel position determining function, blood pressure measurement execution function, and data logging function based on the input data from the ultrasonic probe 6 or the calibration sphygmomanometer 8 connected through the interface circuit 12.

In the example shown in FIG. 1, communication connection between the ultrasonic probe 6 or the calibration sphygmomanometer 8 and the ultrasonic blood pressure measuring device 10 is realized by a cable. However, the communication connection between the ultrasonic probe 6 or the calibration sphygmomanometer 8 and the ultrasonic blood pressure measuring device 10 may be realized by wireless communication by mounting a short-distance radio device 23 or the like on the control board 20. The ultrasonic blood pressure measuring device 10 of the present embodiment can be configured as a portable information terminal, such as a smartphone or a wearable computer capable of executing an application program. Alternatively, the ultrasonic blood pressure measuring device 10 of the present embodiment may be configured as a stationary device or as an external device connected through a mobile phone network, the Internet, or a local area network (LAN).

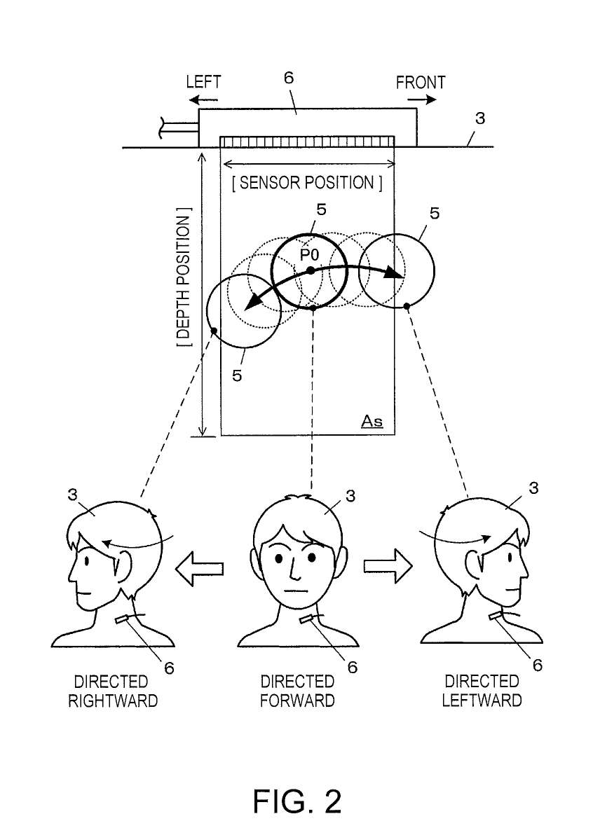

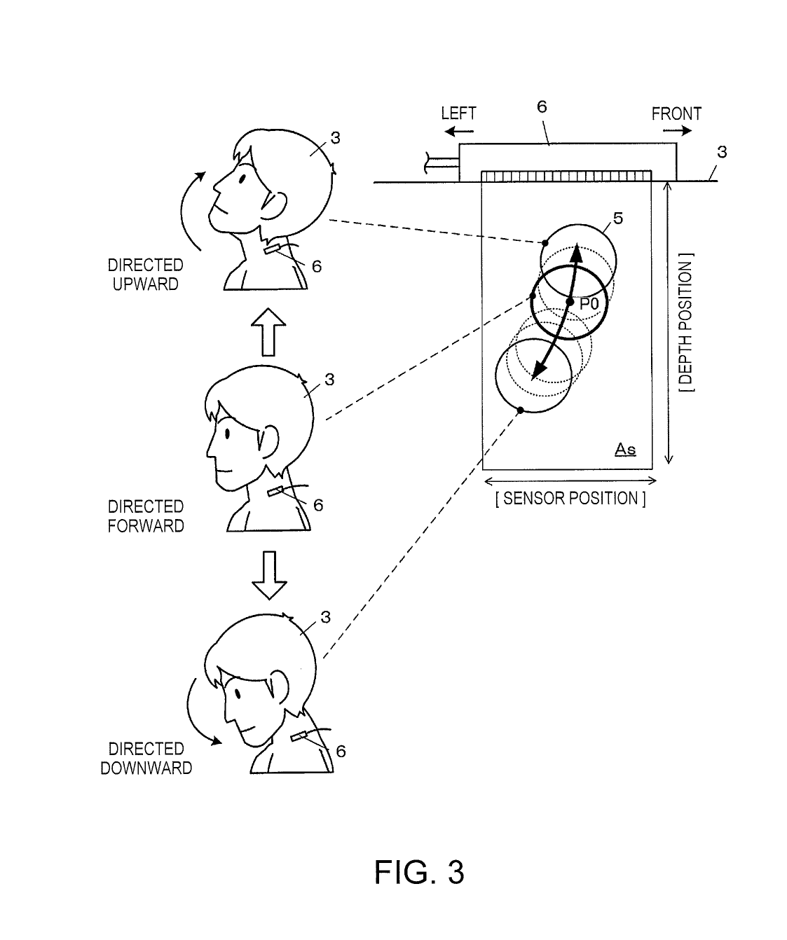

FIGS. 2 and 3 are diagrams showing examples of a change in the blood vessel position of the carotid artery with respect to the ultrasonic probe according to the movement of the neck. FIG. 2 shows a case in which the neck is directed to the left and right, and FIG. 3 shows a case in which the neck is directed up and down.

The blood vessel position is expressed by the coordinate values of two axes of a “depth position” and a “sensor position”. The “depth position” is a position when the traveling direction of the ultrasonic beam from the ultrasonic probe 6 to the inside of the body is set as an axis, and the “sensor position” is a position when the arrangement direction of ultrasonic transducers (ultrasonic sensors) is set as an axis.

In the present embodiment, it is assumed that the blood vessel position is represented by the position of the center of the short-axis section of the blood vessel (blood vessel center position). Needless to say, the front wall (blood vessel wall on a side close to the ultrasonic probe 6) or the rear wall of the blood vessel can also be used. However, since the blood vessel wall continuously expands and contracts due to beating, it is desirable to set the center position as a reference so that the blood vessel positions can be accurately compared. Even if the blood vessel center position is used, the position may change slightly according to the timing of beating. Therefore, when determining the blood vessel position, a position in the diastole (referred to in blood pressure) in which the blood vessel diameter is the minimum or a position in the systole in which the blood vessel diameter is the maximum is adopted.

In the examples shown in FIGS. 2 and 3, since a case is assumed in which the ultrasonic wave is transmitted in a linear direction from the respective ultrasonic transducers of the ultrasonic probe 6, a range that the ultrasonic wave reaches, that is, a scanning range As is a rectangular shape. However, in a case in which the ultrasonic wave can be transmitted in an oblique direction, the scanning range As becomes an approximately trapezoidal shape or fan shape due to the extension of the lower side in FIGS. 2 and 3.

As shown in FIG. 2, assuming that the blood vessel position of the carotid artery 5 in a state in which the person to be measured 3 faces forward is a reference (reference blood vessel position P0), when the person to be measured 3 turns their head to the left and right from the state in which the head is directed to the front, the blood vessel position of the carotid artery 5 is moved in a sensor position direction along the arrangement of the ultrasonic transducers (direction of parallel movement of the relative position with respect to the ultrasonic probe 6) by the expansion and contraction of the skin and the movement of the muscles or the tendon. In addition, as shown in FIG. 3, when the person to be measured 3 moves their head up and down from the state in which the head is directed to the front, the blood vessel position of the carotid artery 5 changes continuously in the depth direction due to the same reasons.

Since the hardness (for example, the stiffness parameter β) of the blood vessel that is the basis for the blood pressure-blood vessel diameter relationship is influenced by such a change in the blood vessel position, a measurement error may occur if the posture when calculating the blood pressure-blood vessel diameter relationship with the hardness of the blood vessel as a constant is different from the posture at the time of measurement.

Therefore, in the present embodiment, the influence of a measurement error due to the posture change of the person to be measured 3 is eliminated by performing the measurement only when the person to be measured 3 is at a specific posture.

The specific posture state in the present embodiment is a state when calculating the blood pressure-blood vessel diameter relationship, and is assumed to be a state in which the head is directed to the front. In addition, the displacement of the blood vessel position is determined at any time with the center of the short-axis section of the blood vessel when calculating the blood pressure-blood vessel diameter relationship as the reference blood vessel position P0. In other words, the relative position of the carotid artery 5 with respect to the ultrasonic probe 6 is determined.

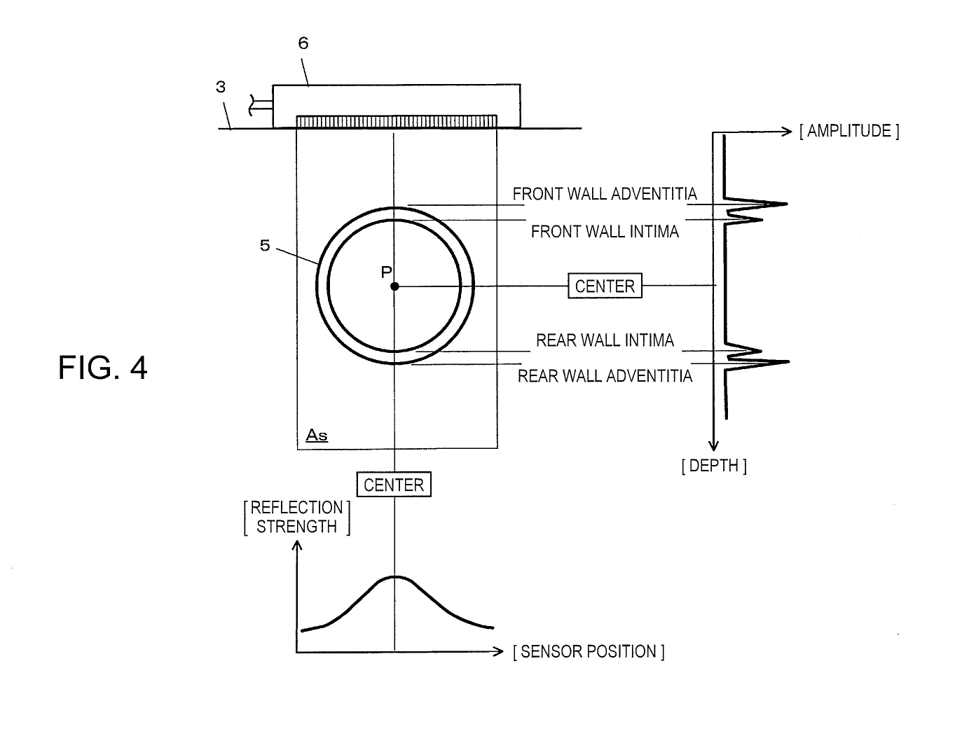

FIG. 4 is a diagram for explaining a method of determining the blood vessel position in the present embodiment.

As a method of detecting the blood vessel center position, the strength of the reception signal of each ultrasonic transducer is used. Specifically, the reflection strength of the transmission wave of the ultrasonic wave increases as the transmission wave of the ultrasonic wave is emitted further perpendicular to the blood vessel section. Accordingly, the position of the ultrasonic transducer that indicates the maximum of the distribution of the reception signal strengths of the respective ultrasonic transducers is assumed to be the sensor position coordinate value of a blood vessel center position P. Then, vascular front and rear walls are detected from the peak position of the signal strength of the amplitude data in the depth direction of the ultrasonic beam at the sensor position, and the intermediate position is assumed to be the depth position coordinate value of the blood vessel center position P.

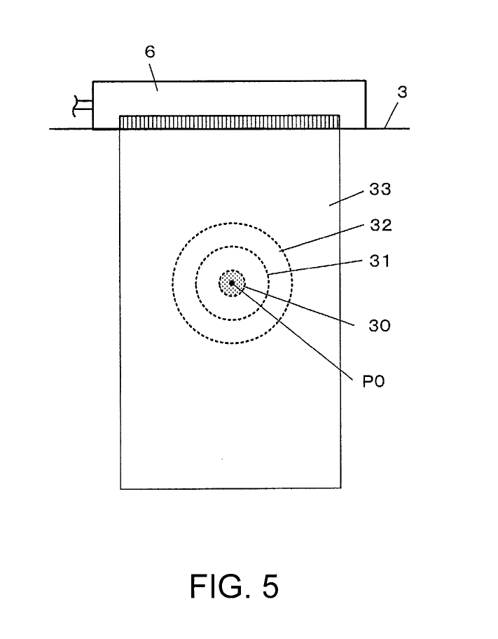

FIG. 5 is a diagram for explaining a method of determining a specific posture state based on the blood vessel position. In the present embodiment, the specific posture state is determined when the position is included in a given measurement allowable blood vessel position range 30 that is set in advance with the reference blood vessel position P0 as a reference point.

The measurement allowable blood vessel position range 30 is set by statistically calculating the allowable range of the influence of the posture change of the person to be measured 3 on the blood pressure measurement result by the pretest.

The shape of the measurement allowable blood vessel position range 30 can be appropriately set. However, as shown in FIGS. 2 and 3, in view of the tendency that the amount of movement of the blood vessel position due to the left and right movement of the neck is larger than the amount of movement of the blood vessel position due to the up-and-down movement of the neck, the shape of the measurement allowable blood vessel position range 30 can be set as an elliptical area that is long in the left and right direction (sensor position direction) and is short in the depth direction.

In other words, if priority is given to measurement accuracy, the measurement allowable blood vessel position range 30 can be set as a true circle area having a radius of approximately 1 millimeter. In this case, since the measurement error can almost be neglected, the measurement allowable blood vessel position range 30 can also be referred to as an “error zero area”. If both versatility and measurement accuracy are required, the measurement allowable blood vessel position range 30 can be set as a region having a short axis of approximately several millimeters and a long axis of approximately several millimeters.

If the blood vessel position determined at each time is outside of the measurement allowable blood vessel position range 30, it is determined that there is a posture change that affects measurement, and blood pressure measurement is not performed. If the blood vessel position determined at each time is within the measurement allowable blood vessel position range 30, the measurement is performed.

In addition, as a larger area surrounding the measurement allowable blood vessel position range 30, it is possible to set an annular small-error area 31, an annular medium-error area 32, and an annular large-error area 33 formed by the scanning range As excluding the small-error area 31 and the medium-error area 32. The assumed measurement error increases stepwise in the relationship of “small-error area<medium-error area<large-error area”. The number of these areas can be appropriately set. Only the measurement allowable blood vessel position range 30 may be set without setting an out-of-range area.

Next, the functional configuration for realizing the present embodiment will be described.

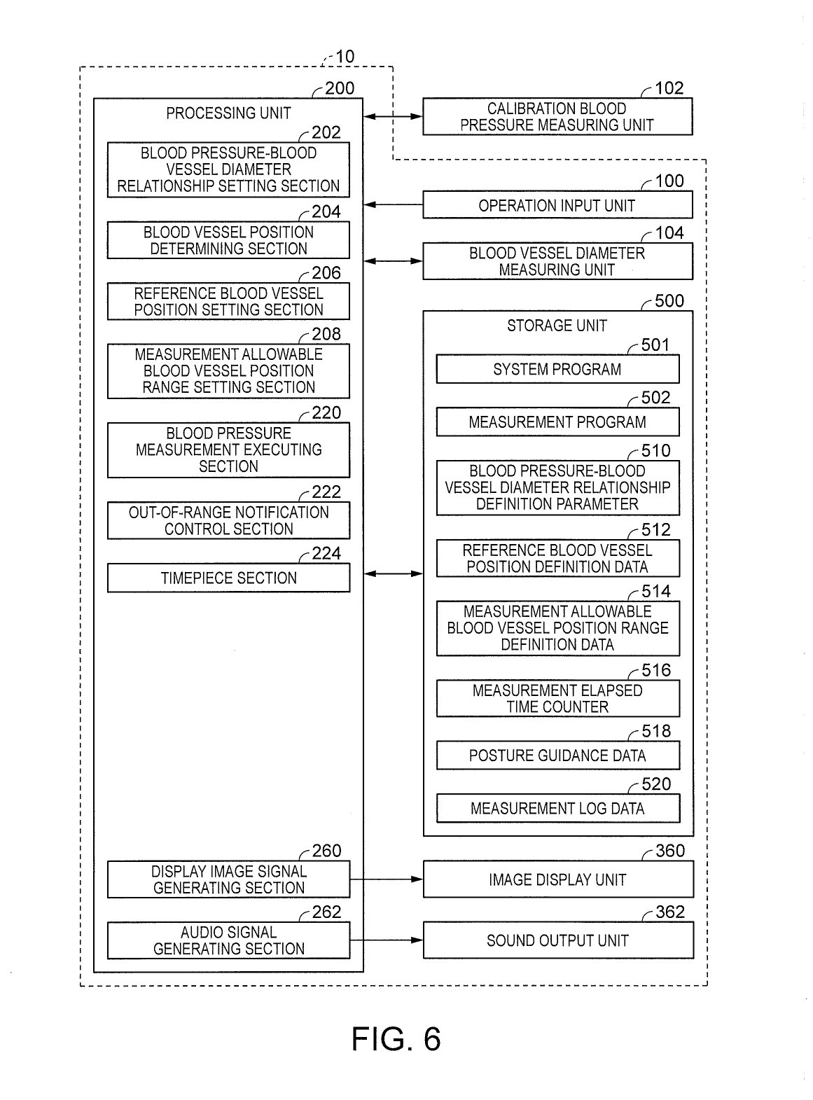

FIG. 6 is a block diagram showing an example of the functional configuration of the ultrasonic blood pressure measuring system 2 of the present embodiment. The ultrasonic blood pressure measuring device 10 includes an operation input unit 100, a calibration blood pressure measuring unit 102, a blood vessel diameter measuring unit 104, a processing unit 200, an image display unit 360, a sound output unit 362, and a storage unit 500.

The operation input unit 100 receives various kinds of operation input performed by the operator, and outputs an operation input signal corresponding to the operation input to the processing unit 200. The operation input unit 100 can be realized by a button switch, a lever switch, a dial switch, a track pad, a mouse, and the like. The touch panel 11 shown in FIG. 1 corresponds to the operation input unit 100.

The calibration blood pressure measuring unit 102 measures calibration blood pressure for calculating the blood pressure-blood vessel diameter relationship, and outputs the measurement result to the processing unit 200. The calibration sphygmomanometer 8 shown in FIG. 1 corresponds to the calibration blood pressure measuring unit 102.

The blood vessel diameter measuring unit 104 is a unit for measuring the blood vessel diameter using an ultrasonic wave. The ultrasonic probe 6 shown in FIG. 1 corresponds to the blood vessel diameter measuring unit 104, and outputs the reflection strength of each ultrasonic transducer, amplitude data in the depth direction, blood vessel diameter, and the like to the processing unit 200.

The processing unit 200 is realized, for example, by a microprocessor, such as a CPU or a graphics processing unit (GPU), or an electronic component, such as an application specific integrated circuit (ASIC) or an IC memory. The processing unit 200 controls the input and output of data between the respective functional units, and performs overall control of the ultrasonic blood pressure measuring device 10 by executing various kinds of arithmetic processing based on a predetermined program or various kinds of data. The ultrasonic blood pressure measuring device 10 shown in FIG. 1 corresponds to the processing unit 200. More specifically, the control board 20 corresponds to the processing unit 200.

The processing unit 200 includes a blood pressure-blood vessel diameter relationship setting section 202, a blood vessel position determining section 204, a reference blood vessel position setting section 206, a measurement allowable blood vessel position range setting section 208, a blood pressure measurement executing section 220, an out-of-range notification control section 222, and a timepiece section 224, a display image signal generating section 260, and an audio signal generating section 262.

As processing relevant to calibration, the blood pressure-blood vessel diameter relationship setting section 202 calculates systolic blood pressure, diastolic blood pressure, systolic blood vessel diameter, and diastolic blood vessel diameter based on the blood pressure obtained by the calibration blood pressure measuring unit 102 and the blood vessel diameter obtained by the blood vessel diameter measuring unit 104, and calculates and sets a blood pressure-blood vessel diameter relationship including the stiffness parameter β. Such a function can be realized by appropriately using a known technique.

The blood vessel position determining section 204 calculates a coordinate value indicating the relative position of a blood vessel to be measured (in the present embodiment, the carotid artery 5: refer to FIG. 1) with respect to the ultrasonic probe 6, that is, a coordinate value indicating the blood vessel center position that represents the blood vessel position, based on the reception signal strength of each ultrasonic transducer and the amplitude data in the depth direction that are continuously obtained by the blood vessel diameter measuring unit 104 (refer to FIG. 4).

The reference blood vessel position setting section 206 sets the reference blood vessel position P0 (refer to FIG. 5). Specifically, the blood vessel position when calculating the blood pressure-blood vessel diameter relationship is adopted.

The measurement allowable blood vessel position range setting section 208 sets the measurement allowable blood vessel position range 30 (refer to FIG. 5). In the present embodiment, a parameter that defines the width of the measurement allowable blood vessel position range 30 including the reference blood vessel position P0 as a reference point is set. However, the operator may be prompted to input the measurement allowable blood vessel position range 30 so that the measurement allowable blood vessel position range 30 can be manually set before the start of measurement.

The blood pressure measurement executing section 220 performs blood pressure measurement based on the relative positional relationship between the ultrasonic probe 6 and the latest blood vessel position at each time. In the present embodiment, when the latest blood vessel position is included in the measurement allowable blood vessel position range 30, control to calculate blood pressure by substituting the data obtained by the blood vessel diameter measuring unit 104 into the blood pressure-blood vessel diameter relationship and store the calculated blood pressure and the measurement date and time in the storage unit 500.

The out-of-range notification control section 222 performs control for notifying that the relative positional relationship between the latest blood vessel position and the ultrasonic probe 6 does not satisfy the measurement allowance conditions. Specifically, when the blood vessel position is outside of the measurement allowable blood vessel position range 30, it is determined that the measurement allowance conditions are not satisfied, and predetermined notification is provided to the image display unit 360. This is referred to as “out-of-range notification”.

The out-of-range notification can be appropriately performed through sound, image display, or the like. In the case of image display, the out-of-range notification may be realized though text display or the display of a predetermined icon, or may be realized by changing the display font on the monitor for the blood pressure or the blood vessel diameter, or changing the font size, or changing the display color of a font or display brightness. Through the out-of-range notification, it is possible to know whether the state in which measurement and recording are not performed is due to the failure of the device or due to the undesirable posture of the person to be measured 3.

The timepiece section 224 counts the passage of time. For example, the timepiece section 224 can be realized by a known timepiece technique, such as a counter using a system clock.

The display image signal generating section 260 is realized, for example, by a processor such as a GPU or a digital signal processor (DSP), a program such as a video signal IC or video CODEC, or an IC memory for drawing frames such as a frame buffer. The display image signal generating section 260 generates an image signal for displaying operation guidance, a measurement result, or the like, and outputs the image signal to the image display unit 360.

The image display unit 360 displays various images based on the image signal input from the display image signal generating section 260. For example, the image display unit 360 can be realized by an image display device, such as a flat panel display, a cathode ray tube (CRT), a projector, or a head-mounted display. In the present embodiment, the touch panel 11 shown in FIG. 1 corresponds to the image display unit 360.

The audio signal generating section 262 is realized, for example, by a digital signal processor (DSP), a processor such as a sound synthesis IC, or an audio CODEC capable of reproducing an audio file. The audio signal generating section 262 generates audio data, such as various operation sounds or voice guidance, and outputs the audio data to the sound output unit 362.

The sound output unit 362 is realized by a device that emits sound based on the audio signal input from the audio signal generating section 262. The speaker 13 shown in FIG. 1 corresponds to the sound output unit 362.

The storage unit 500 is realized by a storage medium, such as an IC memory, a hard disk, or an optical disc, and stores various programs or various kinds of data, such as data during the calculation process of the processing unit 200. In FIG. 1, the IC memory 22 mounted on the control board 20 corresponds to the storage unit 500. In addition, the connection between the processing unit 200 and the storage unit 500 is not limited to a connection using an internal bus circuit in the device, and may be realized by using a communication line, such as a local area network (LAN) or the Internet. In this case, the storage unit 500 may be realized by a separate external storage device from the ultrasonic blood pressure measuring device 10.

The storage unit 500 includes a system program 501, a measurement program 502, a blood pressure-blood vessel diameter relationship definition parameter 510, reference blood vessel position definition data 512, measurement allowable blood vessel position range definition data 514, a measurement elapsed time counter 516, posture guidance data 518, and measurement log data 520. Needless to say, programs or data other than these can also be appropriately stored.

The processing unit 200 executes the system program 501, thereby realizing a basic input/output function as a computer.

The measurement program 502 is executed by the processing unit 200, thereby realizing the blood pressure-blood vessel diameter relationship setting section 202, the blood vessel position determining section 204, the reference blood vessel position setting section 206, the measurement allowable blood vessel position range setting section 208, the blood pressure measurement executing section 220, the out-of-range notification control section 222, and the timepiece section 224, the display image signal generating section 260, and the audio signal generating section 262. When realizing these functional units with hardware, such as electronic circuits, a part of the program for realizing the function can be omitted.

The blood pressure-blood vessel diameter relationship definition parameter 510 includes various parameter values that define the blood pressure-blood vessel diameter relationship using the stiffness parameter β. For example, systolic blood pressure, diastolic blood pressure, systolic blood vessel diameter, diastolic blood vessel diameter, the stiffness parameter β, and the like are included.

In the reference blood vessel position definition data 512, data that defines the reference blood vessel position P0 is stored (refer to FIG. 5). In the present embodiment, a coordinate value indicating the sensor position and a coordinate value indicating the depth position are stored.

In the measurement allowable blood vessel position range definition data 514, a parameter value that defines the measurement allowable blood vessel position range 30 is stored. For example, assuming that the measurement allowable blood vessel position range 30 is an elliptical region, a value of the short axis, a value of the long axis, and the like are stored.

The measurement elapsed time counter 516 shows an elapsed time from the start of measurement.

The posture guidance data 518 is data for guiding the person to be measured 3 to have a specific posture. For example, image data for guidance display or audio data for audio guidance (for example, “please face the front for measurement”) corresponds to the posture guidance data 518.

In the measurement log data 520, measurement results are stored in time series. In the present embodiment, a measurement number automatically assigned in order of measurement, measurement date and time, and a measurement result are stored in time series so as to match each other.

Next, the operation of the ultrasonic blood pressure measuring device 10 will be described.

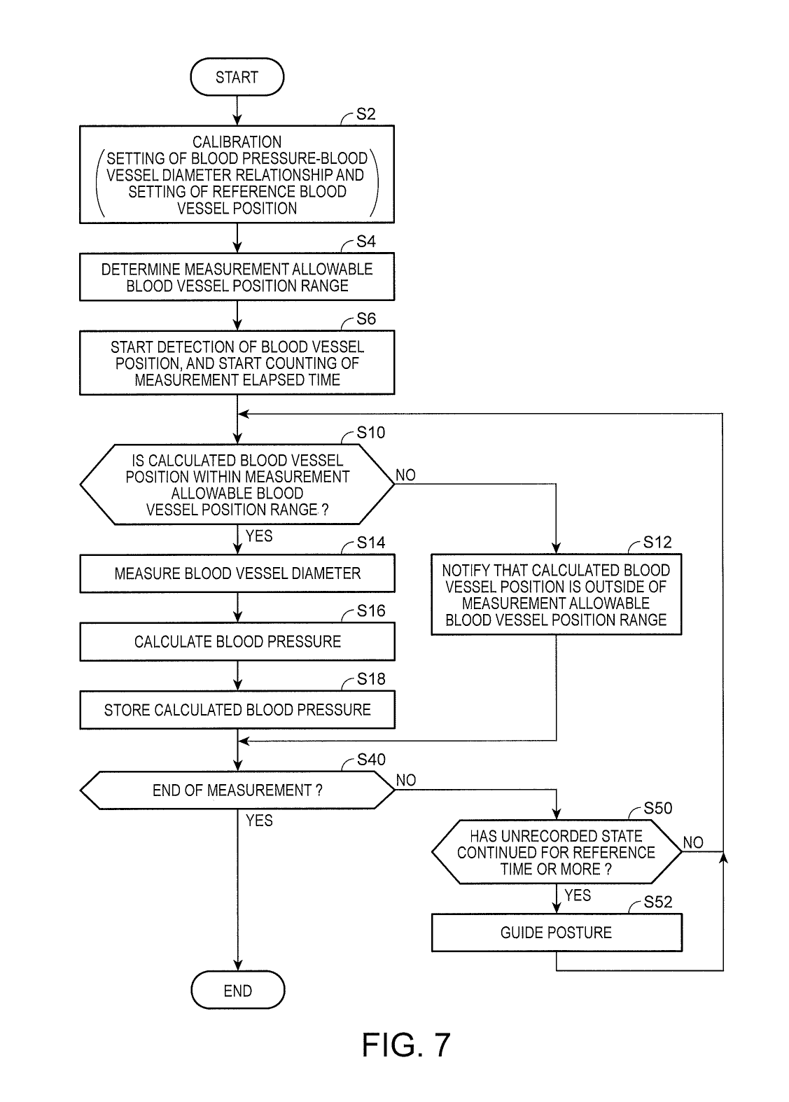

FIG. 7 is a flowchart for explaining the flow of the process related to the blood pressure measurement performed by the ultrasonic blood pressure measuring device 10.

First, the processing unit 200 of the ultrasonic blood pressure measuring device 10 performs calibration processing, sets a blood pressure-blood vessel diameter relationship, and determines the reference blood vessel position P0 (step S2). At the time of calibration, an operation guidance prompting the operator to guide the person to be measured 3 to maintain a state, in which the person to be measured 3 faces the front, for a while is displayed on the touch panel 11. Alternatively, control to emit voice guidance from the speaker 13 may be performed. Since the calibration sphygmomanometer 8 is not necessary when the calibration is completed, it is preferable to appropriately notify that the calibration has been completed.

Then, the ultrasonic blood pressure measuring device 10 sets the measurement allowable blood vessel position range 30 including the reference blood vessel position P0 as a reference point (step S4).

Then, the ultrasonic blood pressure measuring device 10 starts the counting of the measurement elapsed time, and starts the detection of a blood vessel position (step S6). That is, a blood vessel center position is calculated at a timing at which biological information satisfies predetermined conditions (for example, systolic blood pressure or diastolic blood pressure) or at predetermined periods (for example, every second or every beat of the cardiac cycle).

If the calculated blood vessel position is outside of the measurement allowable blood vessel position range 30 (NO in step S10), the ultrasonic blood pressure measuring device 10 provides out-of-range notification without performing measurement (step S12).

On the other hand, if the calculated blood vessel position is within the measurement allowable blood vessel position range 30 (YES in step S10), the ultrasonic blood pressure measuring device 10 measures the blood vessel diameter (step S14), calculates the blood pressure (step S16), and stores the calculated blood pressure in the measurement log data 520 (step S18). When calculating the blood pressure in step S16, a blood vessel diameter used in the detection of the blood vessel position may be used without measuring the blood vessel diameter again in step S14.

Then, it is determined whether or not the predetermined measurement end conditions are satisfied (step S40).

As the measurement end conditions, for example, passage of a predetermined amount of time (for example, passage of 24 hours) from the start of measurement, reaching the upper limit of the number of times of measurement, and detection of a predetermined measurement end operation can be appropriately set.

If the measurement end conditions are satisfied (YES in step S40), the series of processing is ended. If the measurement end conditions are not yet satisfied (NO in step S40), the ultrasonic blood pressure measuring device 10 determines whether or not the unrecorded state has continued for a reference time or more by comparing the measurement elapsed time counter 516 with the measurement date and time of the latest data stored in the measurement log data 520 (step S50). The reference time can be appropriately set according to the measurement purpose.

If the unrecorded state is less than the reference time (NO in step S50), the process returns to step S10. If the unrecorded state has continued for the reference time or more (YES in step S50), the ultrasonic blood pressure measuring device 10 performs posture guidance, which prompts the person to be measured 3 to have a specific posture state for measurement (in the present embodiment, a state in which the head is directed to the front), based on the posture guidance data 518 (step S52), and the process returns to step S10.

As described above, according to the present embodiment, it is possible to manage the measurement so that no measurement is performed when the position of the blood vessel to be measured when calculating the blood pressure-blood vessel diameter relationship before measurement has changed to the extent of causing a measurement error that is not acceptable. That is, it is possible to maintain high measurement accuracy by waiting for the next appropriate measurement chance without performing measurement when the posture of the person to be measured 3 is different from that when calculating the blood pressure-blood vessel diameter relationship.

### MODIFICATION EXAMPLES

Embodiments of the invention are not limited to the above, and constituent components can be appropriately added, omitted, and changed.

### Modification Example 1

For example, although no measurement is performed when the blood vessel position is outside of the measurement allowable blood vessel position range in the embodiment described above, measurement may be performed in a state in which the operator is aware that a measurement error is included. In this case, it is preferable to provide reliability information indicating which degree of error is included in the measurement result together with the measured blood pressure value.

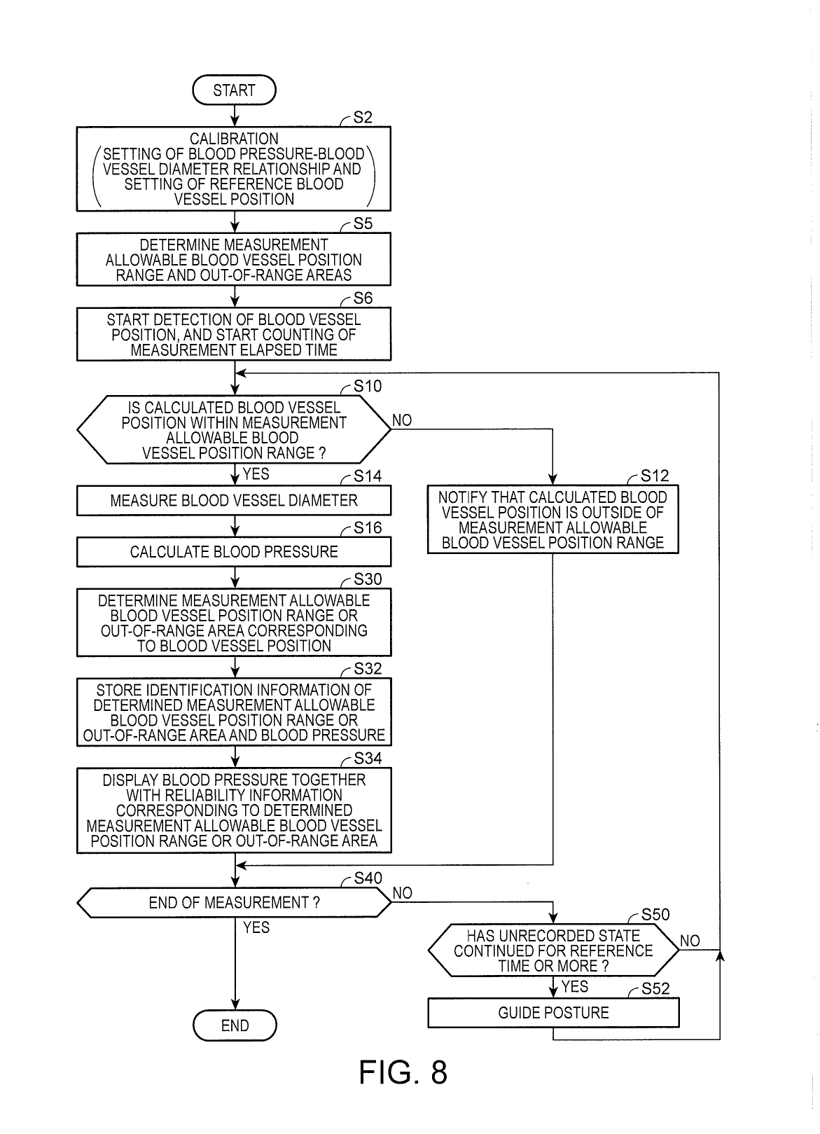

Specifically, instead of step S4 in FIG. 7, out-of-range areas of the small-error area 31, the medium-error area 32, and the large-error area 33 (refer to FIG. 5) are set together with the measurement allowable blood vessel position range 30 (step S5). Then, as shown in FIG. 8, instead of step S18, a measurement allowable blood vessel position range or an out-of-range area corresponding to the latest blood vessel position is determined (step S30). Then, identification information of the determined measurement allowable blood vessel position range or out-of-range area is stored in the measurement log data 520 so as to match the calculated blood pressure (step S32). Then, the blood pressure may be displayed on the touch panel 11 together with reliability information corresponding to the determined measurement allowable blood vessel position range or out-of-range area (step S34).

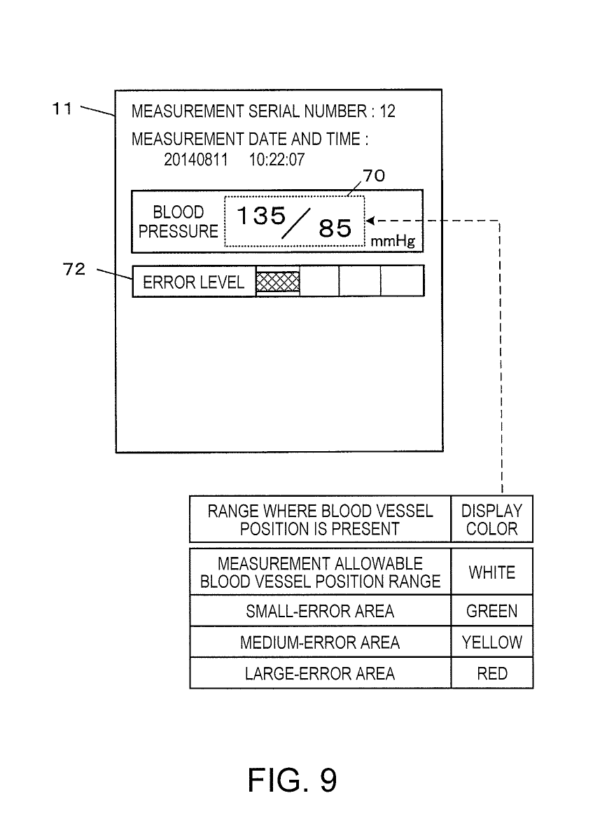

FIG. 9 is a diagram showing a display example of the reliability information in step S34.

The monitor display of blood pressure measurement is performed on the touch panel 11. For example, a blood pressure display 70 after measurement is displayed so as to have a display form difference (in the example shown in FIG. 9, a display color difference) corresponding to the type of the measurement allowable blood vessel position range or the out-of-range area determined in step S32. In addition, the display form difference is not limited to the display color, and may be realized by other elements, such as the size of display text, font type, reversed display, and brightness difference. In addition, error information is displayed as a level gauge 72.

### Modification Example 2

Although the measurement allowable blood vessel position range 30 is set in advance in the embodiment described above, the measurement allowable blood vessel position range 30 may be set according to the person to be measured 3.

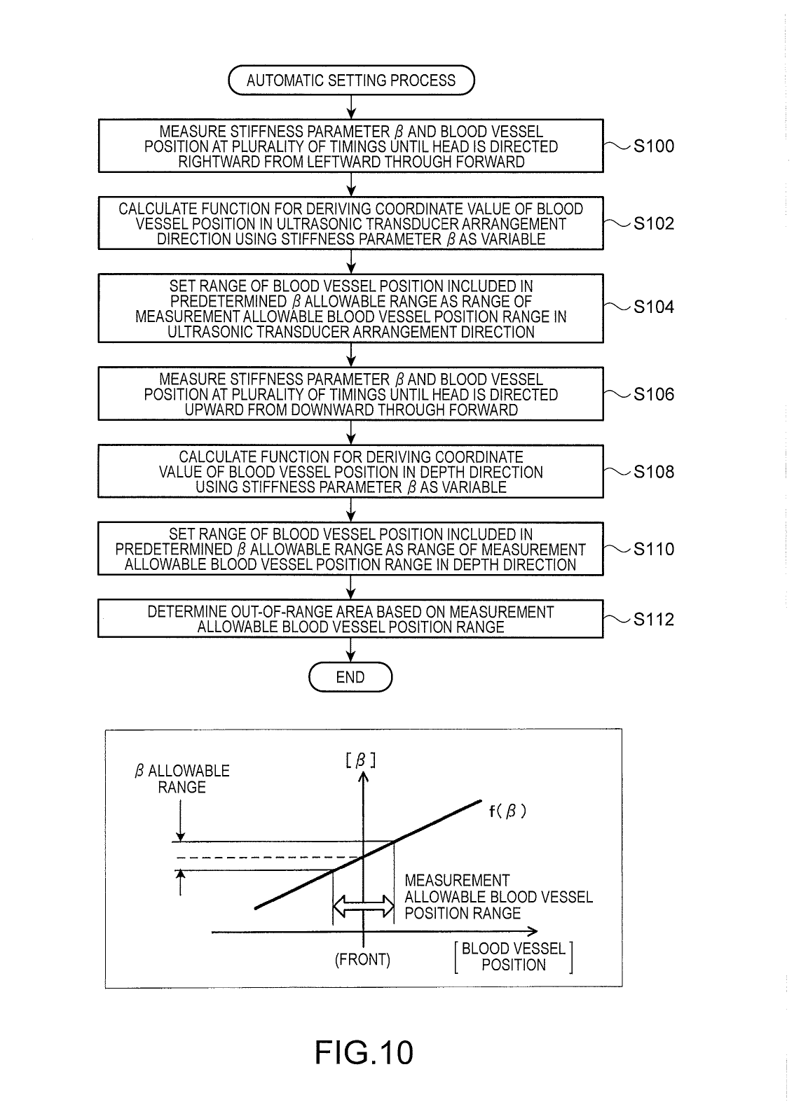

Specifically, instead of step S4 (refer to FIG. 7), an automatic setting process shown in FIG. 10 is performed. In the automatic setting process, first, the person to be measured 3 is made to change their posture until their head is directed to the right from the left through the front, and the stiffness parameter β and the blood vessel position are measured at a plurality of timings during the posture change time (step S100). Then, a function f(β) for calculating the coordinate value of the measured blood vessel position in the ultrasonic transducer arrangement direction using the stiffness parameter β as a variable is calculated (step S102). Postures to be measured may be three of left, front, and right, and a spline function, an approximation curve function, and the like can be appropriately set as the function f(β).

Then, a range of the blood vessel position in the ultrasonic transducer arrangement direction that includes the reference blood vessel position P0, at which the stiffness parameter β falls within a predetermined β allowable range (range of the stiffness parameter β in which an error acceptable in the blood pressure-blood vessel diameter relationship is included), as the center is determined from the function f(β). This is set in the measurement allowable blood vessel position range definition data 514 as a parameter value indicating the range of the measurement allowable blood vessel position range 30 in the ultrasonic transducer arrangement direction (step S104).

The range of the measurement allowable blood vessel position range 30 in the depth position direction is similarly determined.

That is, the person to be measured 3 is made to change their posture until their head is directed upward from downward through the front, and the stiffness parameter β and the blood vessel position are measured at a plurality of timings during the posture change time (step S106). Then, a function g(β) to calculate the depth position of the blood vessel position, which is measured with the stiffness parameter β as a variable, is calculated again (step S108). Then, a range of the blood vessel position in the depth direction that includes the reference blood vessel position P0, at which the stiffness parameter β falls within a predetermined β allowable range (range of the stiffness parameter β in which an error acceptable in the blood pressure-blood vessel diameter relationship is included), as the center is determined from the function g(β). This is set in the measurement allowable blood vessel position range definition data 514 as a parameter value indicating the range of the measurement allowable blood vessel position range 30 in the depth direction (step S110).

Then, an out-of-range area may be set based on the measurement allowable blood vessel position range (step S112).

### Modification Example 3

The method of calculating the blood vessel position is not limited to the example of the embodiment described above, and other methods can be appropriately adopted.

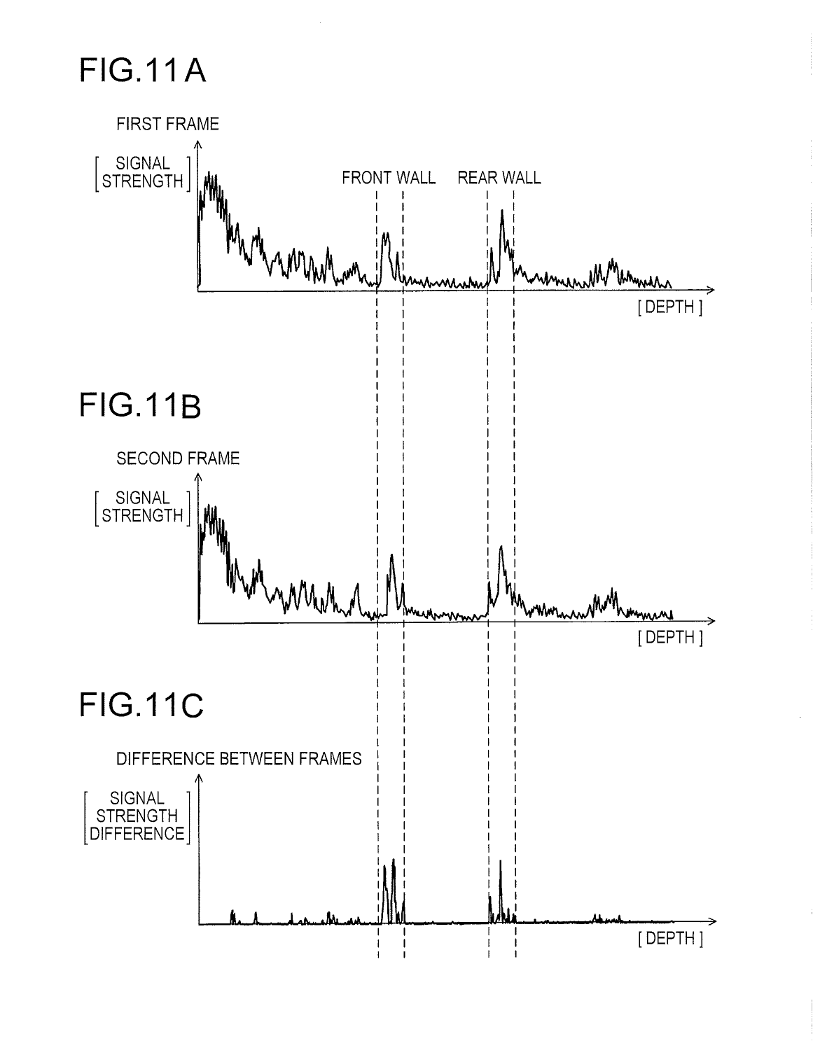

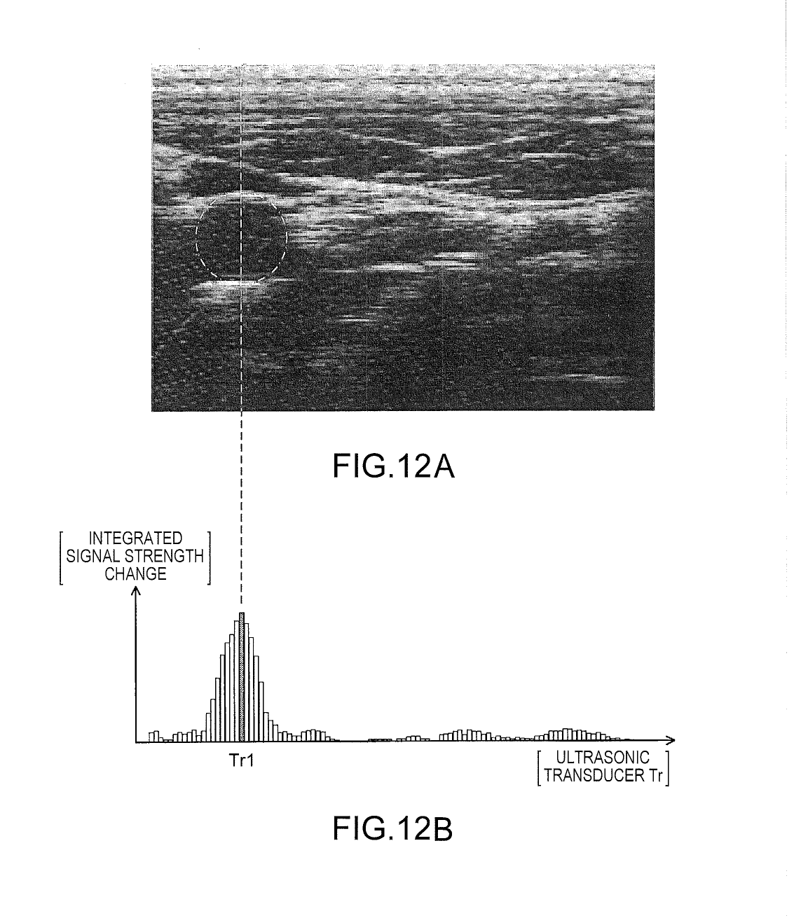

For example, as shown in FIGS. 11A-11C, whenever the reflected wave signals of two adjacent frames (first and second frames in FIGS. 11A and 11B) are obtained, a signal strength difference (difference between the frames in FIG. 11C) is calculated for each depth for each ultrasonic transducer of the ultrasonic probe 6. By repeating this calculation for a predetermined amount of time, the sum of the signal strength differences at all depths is integrated for the respective ultrasonic transducers, as shown in FIGS. 12A and 12B. The peak value of the histogram showing the integrated value of the signal strength differences is searched for, and the ultrasonic transducer corresponding to the peak value magnitude is determined as an “ultrasonic transducer located immediately above the blood vessel”. That is, the ultrasonic transducer indicates the sensor position of the blood vessel center. In addition, as in the embodiment described above, the blood vessel center may be calculated from the positions of vascular front and rear walls.

The entire disclosure of Japanese Patent Application No. 2014-163412 filed on Aug. 11, 2014 is expressly incorporated by reference herein.

What is claimed is:

1. An ultrasonic blood pressure measuring device that measures blood pressure of a blood vessel by performing transmission of an ultrasonic wave and reception of a reflected wave with respect to the blood vessel using an ultrasonic probe, the device comprising:
a blood vessel position determination unit that determines a position of the blood vessel with respect to the ultrasonic probe based on a reception signal of the reflected wave; and
a blood pressure measurement execution unit that executes blood pressure measurement based on a determination result of the blood vessel position determination unit.

2. The ultrasonic blood pressure measuring device according to claim 1,
wherein the blood vessel position determination unit determines the position of the blood vessel according to a center of the blood vessel.

3. The ultrasonic blood pressure measuring device according to claim 1,
wherein the blood pressure measurement execution unit executes blood pressure measurement when the position is included in a measurement allowable blood vessel position range set in advance.

4. The ultrasonic blood pressure measuring device according to claim 3, further comprising:
a notification control unit that performs predetermined notification control when the position is outside of the measurement allowable blood vessel position range.

5. The ultrasonic blood pressure measuring device according to claim 3,
wherein the blood vessel is a carotid artery, and
the measurement allowable blood vessel position range is set such that a range of the position in a direction crossing a transmission direction of the ultrasonic wave with respect to the ultrasonic probe is larger than a range of the position in the transmission direction of the ultrasonic wave with respect to the ultrasonic probe.

6. A blood pressure measuring method, comprising:
performing transmission of an ultrasonic wave and reception of a reflected wave with respect to a blood vessel using an ultrasonic probe and acquiring received data based on a reception signal of the reflected wave;
determining a position of the blood vessel with respect to the ultrasonic probe based on the received data; and
executing blood pressure measurement based on a result of the determination.

7. The blood pressure measuring method according to claim 6,
wherein, in the determination, the position of the blood vessel is determined according to a center of the blood vessel.

8. A blood pressure measuring device, comprising:
a sensor that detects a blood vessel center position and a blood vessel diameter;
a blood pressure measurement execution unit that calculates blood pressure based on the blood vessel diameter detected by the sensor and a relationship between the blood vessel diameter and blood pressure calculated at the time of calibration; and
a display unit that displays the blood pressure calculated by the blood pressure measurement execution unit,
wherein the display unit displays the blood pressure when the blood vessel center position is outside of a predetermined range of the blood vessel center position at the time of calibration.
